(19) United States
(12) Patent Application Publication
(10) Pub. No.: US 2023/0145601 A1
(43) Pub. Date: May 11, 2023

(54) Wearable Article, Assembly, and Method of Making the Same

(71) Applicant: Prevayl Innovations Limited, Manchester, Greater Manchester (GB)

(72) Inventor: Isabel Rose Hepworth, Paris (FR)

(21) Appl. No.: 17/918,219

(22) PCT Filed: Apr. 19, 2021
(86) PCT No.: PCT/GB2021/050927

§ 371 Date: Oct. 11, 2022

(30) Foreign Application Priority Data
Apr. 20, 2020 (GB) 2005694.1
Apr. 20, 2020 (GB) 2005699.0

(51) Int. Cl. A61B 5/00; A61B 5/27

(52) U.S. Cl. CPC A61B 5/6804; A61B 5/27; A61B 2562/227; A61B 5/6831

(57) ABSTRACT

A first layer (301) has an outer surface (302) facing away from the wearer and an inner surface (304) facing towards the wearer. The first layer (301) comprises a recess (303a, 303b). A sensing component (100a, 100b) is attached to he inner surface (304) and comprises fabric base component (101) and first and second conductive regions (109a, 109b, 111a, 111b). The base component (101) has an outer surface (105) facing the inner surface (304) and an inner surface (103) facing towards the wearer. The first conductive region (109a, 109b) is provided on the inner surface (103) and forms an electrode. The second conductive region (111a, 111b) is provided on the outer surface (105). The second conductive region (111a, 111b) is aligned with the recess (303a, 303b) in the first layer of material (301) and forms a connection terminal for connecting with an interface element of an electronics module (200).

### CROSS-REFERENCE TO RELATED APPLICATIONS

This application claims priority from United Kingdom Patent Application number 2005694.1 filed on 20 Apr. 2020 and United Kingdom Patent Application number 2005699.0 filed on 20 Apr. 2020, the whole contents of which are incorporated herein by reference.

### BACKGROUND

Wearable articles comprising sensing components can be designed to interface with a wearer of the article to determine information such as the wearer's heart rate and rate of respiration. The sensing components may comprise electrodes and connection terminals electrically connected together via an electrically conductive pathway. An electronics module for processing and communication can be removably coupled to the connection terminals so as to receive the measurement signals from the electrodes. The wearable articles may be incorporated into or form a garment.

US 2018/0049698A1 discloses a garment manufactured by bonding an adhesive to a first layer of fabric and a second layer of fabric. Holes are cut into each layer of fabric to accommodate the integration of sensors and a mount for a processing unit. Conductive thread embroidered onto a support layer is bonded to the adhesives of the second layer of the fabric. The support layer is removed such that the conductive thread remains bonded to the adhesive. The conductive thread is exposed within each hole, and the mount and sensors can be coupled within the holes such that an electrical connection is established between the mount and at least one sensor via the conductive thread.

It is desirable to overcome at least some of the problems associated with the prior art, whether explicitly discussed herein or otherwise.

### SUMMARY

According to the present disclosure there is provided an assembly, article and method of making the same as set forth in the appended claims. Other features of the invention will be apparent from the dependent claims, and the description which follows.

According to a first aspect of the disclosure, there is provided an assembly. The assembly comprises an electronics module comprising a housing and an interface element provided on an outside surface of the housing. The assembly comprises a wearable article comprising a first layer of flexible material. The first layer of material comprises a recess bounded by an edge of the first layer of material. The recess is arranged to receive at least part of the interface element to seat the electronics module on a surface of the first layer of material. The recess is sized to restrict movement of the electronics module along the surface of the first layer of material.

Advantageously, the recess restricts movement of the electronics module along the surface of the first layer of material. The recess is bounded by the flexible material. This prevents the electronics module from sliding relative to the first layer of material due to motion of a wearer wearing the article. The sliding or other movement of the electronics module could cause the electronics module to move out of a desired position such as an optimum position for communication with sensing components provided in the wearable article. Moreover, the recess facilitates correct location of the electronics module on the surface of the first layer of material.

The recess may act as a guide or keying mechanism to enable the electronics module to be located in the desired position on the first layer of material. Metal or other mechanical fasteners are not required to hold the electronics module in place.

The edge may form a barrier that is arranged to abut against at least part of the interface element when received within the recess to restrict movement of the electronics module along the surface of the first layer of material.

Movement of the electronics module along the surface of the first layer of material may cause at least part of the interface element to abut against the barrier such that further movement of the electronics module along the surface of the first layer of material is restricted.

At least part of the edge of the first layer of material may have a profile which corresponds to the profile of at least part of the interface element arranged to be received within the recess.

At least part of the edge of the first layer of material may have an arcuate profile which corresponds to an arcuate profile of at least part of the interface element arranged to be received within the recess.

At least part of the edge of the first layer of material may have a straight profile which corresponds to a straight profile of at least part of the interface element arranged to be received within the recess

The recess may have a complementary shape to at least part of the interface element arranged to be received within the recess.

The recess may be sized to restrict axial movement of the electronics module about a longitudinal axis of the electronics module. The recess may therefore stop rotational movement of the electronics module.

The edge may form a barrier that is arranged to abut against at least part of the interface element when received within the recess to restrict axial movement of the electronics module about the longitudinal axis of the electronics module.

Axial movement of the electronics module about the longitudinal axis of the electronics module may cause at least part of the interface element to abut against the barrier such that further movement of the electronics module along the surface of the first layer of material is restricted.

The electronics module may comprise a plurality of interface elements. The first layer of material may comprise a single recess for receiving the plurality of interface elements. The first layer of material may comprise a plurality of recesses for receiving the plurality of interface elements.

The electronics module may comprise a number N of interface elements and the first layer of material may comprise a corresponding number N of recesses. Each of the N recesses may be arranged to receive at least part of one of the interface elements to seat the electronics module on a surface of the first layer of material. N may be a number greater than or equal to 2. The number N may correspond to the number of connection terminals provided within the wearable article.

The wearable article may further comprise an attachment mechanism for releasably attaching the electronics module to the wearable article. The attachment mechanism may restrict movement of the electronics module away from the surface of the first layer of material. The attachment mechanism may be separate to the recess and may located apart from the recess and not within the recess.

The attachment mechanism may apply pressure to the electronics module to urge the electronics module towards the surface of the first layer of material.

The attachment mechanism may comprise a pressure membrane which is disposed over the electronics module when the electronics module is seated on the surface of the first layer of material.

The pressure membrane may comprise a second layer of material. The second layer of material may be attached to the first layer of material to form a pocket space in which the electronics module may be located. The second layer of material may comprise an elastomeric material. The second layer of material may be a fabric layer that comprises an elastomeric material.

The recess may be provided within the pocket space.

The attachment mechanism may comprise a magnet arranged to cooperate with a magnet mounted within the electronics module to releasably attach the electronics module to the wearable article.

The wearable article may comprise a sensing component. The electronics module may be communicatively connected to the sensing component when seated on the surface of the first layer of material

The interface element may be a conductive element. The interface element may form an electrical connection with the sensing component when the interface element is seated in the recess.

The electronics module may be arranged to receive signals from and/or send signals to the sensing component via the interface element.

The recess may have an opening that extends through the first layer of material. The interface element may extend at least partially through the opening to form the electrical connection with the sensing component.

The sensing component may comprise an electrode arranged to monitor activity from a body surface of the wearer.

The first layer of material may be a fabric layer. The first layer of material may be a waterproof layer.

According to a second aspect of the disclosure, there is provided a wearable article. The wearable article comprises a first layer of material having an outer surface facing away from the wearer when worn and an inner surface facing towards the wearer when worn. The first layer of material comprises a recess provided in the outer surface. The wearable article comprises a sensing component attached to the inner surface of the first layer of material, the sensing component has an outer surface facing the inner surface of the first layer of material and an inner surface facing towards the wearer when worn. The sensing component comprises a first conductive region provided on the inner surface and arranged to form an electrode for monitoring activity at a body surface of the wearer when worn, and a second conductive region provided on the outer surface and arranged to form a connection terminal for connecting with an interface element of an electronics module.

Advantageously, the article provides a sensing component with first and second conductive regions provided on opposing surfaces of a sensing component. The sensing component is attached to an inner surface of the first layer of material. The first layer of material has a recess which enables an electronics module seated on the outer surface of the first layer of material to form a communication connection with the connection terminal. The wearable article improves the utility of the article as it enables the communication connection to be formed between the two opposing surfaces of the sensing component. This improves the mechanism by which an electronics module can be connected to an electrode of the article.

The sensing component may comprise a fabric base component having the outer surface and the inner surface. The first conductive region may be provided on the inner surface of the base component. The second conductive region may be provided on the outer surface of the base component.

Advantageously, a one-piece sensing component can be provided that includes the electrode, connection terminal. These conductive regions are provided on a common fabric base component. This simplifies the manufacture and assembly of the wearable article as the sensing component can be manufactured separately and then simply disposed on the inner surface of the first layer of the material to provide the desired sensing functionality for the wearable article. A separate electrode and connection terminal are not required to be positioned on external surfaces of the garment and then connected to an internal conductive thread network.

The recess may be sized such that an interface element of an electronics module may extend at least partially into the recess to connect with the connection terminal.

The connection terminal may be aligned with the recess in the first layer of material.

The recess may be an opening that extends through the first layer of material from the outer surface to the inner surface.

The connection terminal may extend at least partially through the opening.

The sensing component may comprise a gripper component. The gripper component may be provided on the inner surface of the sensing component. The gripper component may be arranged to grip the sensing component to the body surface. The gripper component may provide a high friction or non-slip surface.

The gripper component provides additional grip between the article and the wearer's body surface. This additional grip reduces movement of the electrode on the skin surface. The reduction in movement results in a reduction of movement induced electrical noise appearing in signals provided by the electrodes. Furthermore, the gripper component may encourage perspiration or retain moisture in the vicinity of the electrode which can improve the sensing of electrical signals by the electrode.

The gripper component may be provided in the vicinity of the first conductive region. The gripper component may be provided around at least part of the periphery of the first conductive region.

The gripper component may be a raised section that extends away from the inner surface of the sensing component.

The article may further comprise a filler material disposed within the gripper component. The filler material may comprise an expanding yarn.

The filler material may serve a stabilising function for the gripper component. The filler material may raise the profile of the gripper component out from the sensing component and increase the quality, consistency and area of its contact against the skin surface. This is provided without requiring an increase in the amount of compression applied to the skin surface by the article. Moreover, the expanding yarn can be integrally knit with the remainder of the sensing component which simplifies the manufacturing process and avoids the need to separately insert filler material after the article is formed.

The gripper component may comprise silicone or other material having similar gripping properties. The gripper component may comprise a silicone coating applied to at least part of the first surface of the base component. The silicone component may be selectively applied to the first surface of the base component such as to coat the base component around one or more edges of the first conductive region.

The gripper component may comprise a silicone tape applied to the first surface of the base component.

In some implementations, the gripper component is between 0.5 mm and 30 mm wide.

The gripper component may comprise a gripping yarn such as a silicone yarn.

The first and/or the second conductive regions may be wider than the conductive pathway.

Advantageously, the electrode may be wider than the conductive pathway. Having a wider electrode is beneficial in providing increased surface area of electrode contact with the skin surface. Having a narrower conductive pathway is beneficial in terms of improving comfort for the wearer and minimising the visual appearance of the sensing component on the article. The connection terminal may also be wider than the conductive pathway. Having a wider connection terminal is beneficial in terms of improving the electrical connection between the connection terminal and the interface element of the electronics module.

The first conductive region may extend away from the inner surface to form a raised first conductive region.

The first conductive region may extend from the inner surface to a greater extent than the gripper component.

The second conductive region may extend away from the outer surface to form a raised second conductive region.

The first conductive region and/or second conductive region may extend to a height of between 0.2 mm and 30 mm from the inner surface. The first conductive region and/or second conductive region may extend to a height of between 0.2 mm and 25 mm, 0.2 mm and 20 mm, 0.2 mm and 15 mm, 0.2 mm and 10 mm, 0.2 mm and 5 mm, 0.2 mm and 2 mm, 0.2 mm and 1 mm and 0.2 mm and 0.5 mm. The first conductive region and/or second conductive region may extend to a height of between 0.5 mm and 30 mm, 1 mm and 30 mm, 2 mm and 30 mm, 5 mm and 30 mm, 10 mm and 30 mm, 15 mm and 30 mm, 20 mm and 30 mm, and 25 mm and 30 mm. In some examples, the first conductive region and/or second conductive region extends to a height of between 2 mm and 5 mm.

The conductive regions may be formed from conductive yarn. The conductive regions may be a woven or knitted component. The conductive regions may be formed from a single length of conductive yarn during a single knitting operation. This may mean that the first conductive region, second conductive region, and conductive pathway are formed from the same conductive yarn during a single knitting operation.

The sensing component may comprise a base component that defines the inner and outer surfaces. The base component may be a fabric component. The base component may comprise a non-conductive fabric layer. The base component may be a woven or knitted component. The conductive regions may be integrally formed with the base component so as to form an article of a unitary construction that comprises the base component and conductive regions. This simplifies the process of manufacturing the sensing component and means that separate conductive elements do not need to be provided and individually attached to the base component.

The first conductive region may be wider than the second conductive region. The second conductive region may be wider than the first conductive region. The first and second conductive regions may have the same width.

The first and/or second conductive region may be tapered. This may mean that the first and/or second conductive region gradually increases in height. This may help reduce any potential fraying such as when an electronics module is connected to and removed from a conductive region functioning as a connection terminal.

An assembly comprising the electronics module and the wearable article is also provided.

According to a third aspect of the disclosure, there is provided a method of manufacturing a wearable article. The method comprises providing a first layer of material having an outer surface facing away from the wearer when worn and an inner surface facing towards the wearer when worn, the first layer of material comprises a recess provided in the outer surface. The method comprises attaching a sensing component to the inner surface of the first layer of material, the sensing component has an outer surface facing the inner surface of the first layer of material and an inner surface facing towards the wearer when worn, the sensing component comprises a first conductive region provided on the inner surface and arranged to form an electrode for monitoring activity at a body surface of the wearer when worn, and a second conductive region provided on the outer surface and arranged to form a connection terminal for electrically connecting with an interface element of an electronics module.

The recess may be sized such that an interface element of an electronics module may extend at least partially through the recess.

The wearable article may be the wearable article of the second aspect of the disclosure.

According to a fourth aspect of the disclosure, there is provided a wearable article. The wearable article comprises a first layer of material. The wearable article comprises a second layer of material positioned external to the first layer of material and attached to the first layer of material to define a pocket space for receiving an electronics module. The wearable article comprises an elastomeric material provided between the first and second layers of material and arranged to tension the wearable article when worn. The elastomeric material defines an open region in the pocket space for receiving the electronics module.

Advantageously, the elastomeric material provides tensioning for the wearable article while still providing an open region in the pocket space for receiving the electronics module. This means that the elastomeric material does not affect the placement of the electronics module in the pocket space and does not affect any connections formed between the electronics module and other components of the wearable article.

The elastomeric material may comprise a first end and a second end. The first end and second ends may be spaced apart from one another to define the open region in the pocket space.

The first end may be attached to one or both of the first and second layers of material at a first edge of the pocket space. The second end may be attached to one or both of the second layers of material at a second edge of the pocket space.

The elastomeric material may form a continuous strip of material that extends from the first end to the second end.

The elastomeric material may comprise an opening that forms the open region in the pocket space. The elastomeric material may form a continuous loop of material.

The elastomeric material, when worn, may extend around at least part of the wearer of the wearable article.

The first layer of material, second layer of material, and elastomeric material may form a band which, when worn, extends around the circumference of at least part of the wearer of the wearable article.

The band may be a waist band arranged to extend around the waist of the wearer.

The band may be an arm band arranged to extend around the arm of the wearer.

The band may be a chest band arranged to extend around the chest of the wearer.

The wearable article may be a bra comprising a front portion, a back portion, and the band. The band may be an underband of the bra that extends from a lower margin of the front portion and the back portion of the bra.

The wearable article may further comprise a sensing component. The electronics module may be able to communicate with the sensing component through the open region when positioned in the pocket space.

The first layer of material may comprises a recess arranged to receive at least part of the interface element of the electronics module so that the electronics module may communicate with the sensing component, wherein the recess is aligned with the open region formed by the elastomeric material.

An assembly comprising the electronics module and the wearable article is also provided.

According to fifth aspect of the disclosure, there is provided a method of manufacturing a wearable article. The method comprises providing a first layer of material and a second layer of material. The method comprises disposing an elastomeric material between the first and second layers of material, wherein the elastomeric material is arranged to tension the wearable article when worn. The method comprises attaching the second layer of material to the first layer of material to form a pocket space for receiving an electronics module, wherein the elastomeric material defines an open region in the pocket space for receiving the electronics module.

The wearable article may be the wearable article of the fourth aspect of the disclosure.

According to a sixth aspect of the present disclosure, there is provided a wearable article. The wearable article comprises a first layer of material having an outer surface facing away from the wearer when worn and an inner surface facing towards the wearer when worn. The wearable article comprises a waterproof layer positioned on the outer surface of the first layer of material, the waterproof layer having a recess arranged to receive at least part of an interface element of an electronics module, wherein the recess is sized such that, when the interface element is positioned in the recess, ingress of water through the recess is restricted.

Advantageously, the waterproof layer prevents against water ingress when the electronics module is positioned on the first layer of material and the conductive pads are positioned in the recesses. Because the interface element forms a tight fit with the recess, water ingress through the recess is restricted. Moreover, the waterproof layer prevents water ingress through the potentially water permeable first layer of material. As a result, moisture such as sweat from the wearer is restricted from traversing through the first layer of material to reach the electronics module. This helps protect the electronics module against damage due to water ingress. In addition, this helps protect against water in forming an electrical short by the interface of the electronics module.

The recess may be bounded by an edge of the waterproof layer. The edge may be arranged to abut against at the interface element when received within the recess so as to restrict the ingress of water through the opening.

The recess may be bounded by an edge of the waterproof layer. At least part of the edge may have a profile which corresponds to the profile of at least part of the interface element arranged to be received within the recess.

At least part of the edge of the waterproof layer may have an arcuate profile which corresponds to an arcuate profile of at least part of the interface element arranged to be received within the recess.

At least part of the edge of the waterproof layer may have a straight profile which corresponds to a straight profile of at least part of the interface element arranged to be received within the recess

The recess may have a complementary shape to at least part of the interface element arranged to be received within the recess.

The waterproof layer may comprise a single recess for receiving a plurality of interface elements of the electronics module.

The waterproof layer may comprise a plurality of recesses for receiving a plurality of interface elements.

The electronics module may comprise a number N of interface elements and the waterproof layer may comprise a corresponding number N of recesses. Each of the N recesses may be arranged to receive at least part of one of the interface elements, and wherein N is a number greater than or equal to 2.

The first layer of material may comprises a recess provided in the outer surface, wherein the recess is sized to receive at least part of the interface element of the electronics module, and wherein the recess in the waterproof layer is an opening which extends through the waterproof layer and is aligned with the recess in the first layer of material.

The recess in the first layer of material may have a complementary size and/or shape to the opening in the waterproof layer.

An assembly comprising the electronics module and the wearable article is also provided.

According to a seventh aspect of the present disclosure, there is provided an article. The article comprises a base component having a first surface and a second surface opposing the first surface. The article further comprises a sensing component attached to the base component. The sensing component comprises: a first conductive region provided on a first surface of the base component to form an electrode for monitoring activity at a body surface; a second conductive region provided on the second surface of the base component and arranged to form a connection terminal for electrically connecting with an electronics module; and a conductive pathway electrically connecting the first conductive region to the second conductive region. The article further comprises a gripper component provided on a surface of the base component.

The gripper component maybe provided on the first surface of the base component, wherein the gripper component is arranged to grip the article to the body surface.

Advantageously, the article provides a sensing component with first and second conductive regions provided on opposing surfaces of a base component. This improves the utility of the article as it enables an electrical connection to be formed between the two opposing surfaces of the base component. This improves the mechanism by which an electronics module can be electrically connected to an electrode of the article.

Advantageously still, the article further comprises a gripper component that provides a grippy, high friction, non slip surface for the article. In preferred examples the gripper component is arranged to grip the article to the body surface. The gripper component provides additional grip between the article and the wearer's body surface. This additional grip reduces movement of the electrode on the skin surface. The reduction in movement results in a reduction of movement induced electrical noise appearing in signals provided by the electrodes. Furthermore, the gripper component may encourage perspiration or retain moisture in the vicinity of the electrode which can improve the sensing of electrical signals by the electrode.

The gripper component may be provided in the vicinity of the first conductive electrode. The gripper component may be provided around at least part of the periphery of the first conductive region.

The gripper component may be a raised section that extends away from the first surface of the base component.

The article may further comprise a filler material disposed within the gripper component. The filler material may comprise an expanding yarn. The filler material may comprise a plurality of tuck stitches of the expanding yarn. The filler material may comprise a plurality of float and tuck stitches of the expanding yarn.

The filler material may serve a stabilising function for the gripper component. The filler material may raise the profile of the gripper component out from the base component and increase the quality, consistency and area of its contact against the skin surface. This is provided without requiring an increase in the amount of compression applied to the skin surface by the article.

Moreover, the expanding yarn can be integrally knit with the remainder of the article which simplifies the manufacturing process and avoids the need to separately insert filler material after the article is formed.

The gripper component may comprise silicone. The gripper component may comprise a silicone coating applied to at least part of the first surface of the base component. The silicone component may be selectively applied to the first surface of the base component such as to coat the base component around one or more edges of the first conductive region.

The gripper component may comprise a silicone tape applied to the first surface of the base component.

In some implementations, the gripper component is between 0.2 mm and 30 mm wide. The gripper component may comprise a gripping yarn such as a silicone yarn.

The first and/or the second conductive regions may be wider than the conductive pathway.

Advantageously, the electrode is wider than the conductive pathway. Having a wider electrode is beneficial in providing increased surface area of electrode contact with the skin surface.

Having a narrower conductive pathway is beneficial in terms of improving comfort for the wearer and minimising the visual appearance of the sensing component on the article. The connection terminal is also wider than the conductive pathway. Having a wider connection terminal is beneficial in terms of improving the electrical connection between the connection terminal and the interface element of the electronics module.

The first conductive region may extend away from the first surface of the base component to form a raised first conductive region.

The first conductive region may extend from the first surface to a greater extent than the gripper component.

The second conductive region may extend away from the second surface of the base component to form a raised second conductive region.

The first conductive region and/or second conductive region may extend to a height of between 0.2 mm and 30 mm from the first surface of the base component. The first conductive region and/or second conductive region may extend to a height of between 0.2 mm and 25 mm, 0.2 mm and 20 mm, 0.2 mm and 15 mm, 0.2 mm and 10 mm, 0.2 mm and 5 mm, 0.2 mm and 2 mm, and 0.2 mm and 1 mm. The first conductive region and/or second conductive region may extend to a height of between 0.5 mm and 1 mm, 1 mm and 30 mm, 2 mm and 30 mm, 5 mm and 30 mm, 10 mm and 30 mm, 15 mm and 30 mm, 20 mm and 30 mm, and 25 mm and 30 mm. In some examples, the conductive region extends to a height of between 2 mm and 5 mm.

The conductive pathway may extend along a surface of the base component. The conductive pathway may extend along the first surface of the base component. The conductive pathway may be flush with a surface of the base component. The conductive pathway may extend at least partially within the base component.

The first conductive region and/or the second conductive region may extend away from the base component to a greater extent than the conductive pathway.

The conductive pathway may extend through the base component to electrically connect the first conductive region to the second conductive region.

The sensing component may be formed from conductive yarn.

The sensing component may be a woven or knitted component.

The sensing component may be formed from a single length of conductive yarn during a single knitting operation. This may mean that the first conductive region, second conductive region, and conductive pathway are formed from the same conductive yarn during a single knitting operation. This simplifies the manufacturing process and increases the comfort of the article as elements such as wires and hardware connectors are not required. Further, as the sensing component is knitted, the sensing component is able to stretch with the base component without the electrical properties (e.g. the resistivity) of the sensing component being affected. This is because when a knitted article is stretched, the yarn is not directly stretched, but rather the stitches are deformed. This contrasts with woven articles were the yarns are directly stretched when the woven article is stretched. It will be appreciated that stretching a conductive yarn can change its electrical properties.

The base component may be a woven or knitted component.

The sensing component may be integrally formed with the base component so as to form an article of a unitary construction that comprises the base component and sensing component. This may mean that the article forms an all-in-one, integrated, wearable article. The sensing component can be produced at the same time, together, using the same knitting or weaving machine. This simplifies the process of manufacturing the article and means that separate conductive elements do not need to be provided and individually attached to the base component.

The first conductive region may be wider than the second conductive region.

The article may be a wearable article. The wearable article may be a garment.

The article may be arranged to be integrated into a wearable article. The wearable article may be a garment. The article may be arranged to be stitched, bonded or otherwise adhered to a wearable article.

The first and/or second conductive region may be tapered. This may mean that the first and/or second conductive region gradually increases in height. This may help reduce any potential fraying such as when an electronics module is connected to and removed from a conductive region functioning as a connection terminal.

The article may comprise a plurality of sensing components. This may simplify the manufacturing process as it is not required to manufacture individual articles each comprising only one sensing component.

The article may be arranged to be separated into a plurality of separate articles, wherein each of the plurality of separate articles comprises at least one sensing component. The article may comprise perforations and/or drawthread to facilitate the separation of the article. This enables a single article comprising a number of sensing components to be manufactured and subsequently separated to form a desired number of smaller articles each comprising one or more sensing components. One or more of the separated articles may comprise more than one sensing component. One or more of the separated articles may comprise two or more, five or more or even 10 or more sensing components.

The sensing components may be arranged on the base component to form a plurality of rows of sensing components. The rows may be arranged to be separated from one another to form the plurality of separate articles.

The plurality of sensing components may be arranged in a line on the article.

The plurality of sensing components may be arranged to form at least one pair of sensing components. For each pair of sensing components, the second conductive regions may be proximate to one another, and the first conductive regions may be spaced apart from one another.

The plurality of sensing components may comprise at least two sensing components of different lengths.

The article may comprise an information element for indicating the length of at least one of the sensing components. The information element may be printed, stitched or otherwise incorporated into the article.

The article may comprise a number N of sensing components. N may be greater than 2. N may be 5 or more. N may be 10 or more. N may be 100 or more.

All of the sensing components may be formed from a single length of conductive yarn. The sensing components may therefore be interconnected. Separating the articles may remove the interconnection between different sensing components.

The article may be a flexible article comprising a flexible base component. The article may be a fabric article comprising a fabric base component.

According to an eighth aspect of the present disclosure, there is provided a method of manufacturing an article. The method comprises forming a base component having a first surface and a second surface opposing the first surface. The method comprises forming a sensing component attached to the base component, wherein the sensing component comprises a first conductive region provided on the first surface of the base component; a second conductive region provided on the second surface of the base component, and a conductive pathway electrically connecting the first conductive region to the second conductive region. The method comprises forming a gripper component on a surface of the base component. The gripper component may be on the first surface of the base component, wherein the gripper component is arranged to grip the article to the body surface.

The article may be the article of the seventh aspect of the disclosure. There is also provided an article manufactured according to the method of the eighth aspect of the disclosure.

The wearable articles of any of the aspects of the disclosure described above may comprise any or all of the features of the wearable articles of the other aspects of the disclosure.

### BRIEF DESCRIPTION OF THE DRAWINGS

Examples of the present disclosure will now be described with reference to the accompanying drawings, in which:

FIG. 1 shows an exploded view of an example assembly according to aspects of the present disclosure;

FIG. 2 shows perspective view of the assembly of FIG. 1;

FIG. 3 shows the outer surface of the assembly of FIG. 1;

FIG. 4 shows the inner surface of the assembly of FIG. 1;

FIG. 5 shows a perspective view of the electronics module of the assembly of FIG. 1;

FIG. 6 shows the inner surface of the electronics module of FIG. 5;

FIG. 7 shows a perspective view of the first layer of material of the assembly of FIG. 1;

FIG. 8 shows the electronics module of FIG. 5 seated on the first layer of material of FIG. 7;

FIG. 9 shows the inner surface of the assembly of FIG. 8;

FIG. 10 shows a cross-sectional view of the assembly of FIG. 8;

FIG. 11 shows a perspective view of one of the sensing components of FIG. 1;

FIG. 12 shows the inner surface of the sensing component of FIG. 11;

FIG. 13 shows the outer surface of the sensing component of FIG. 11;

FIG. 14 shows a side view of the sensing component of FIG. 11;

FIG. 15 shows a simplified exploded view of the assembly of FIG. 1;

FIG. 16 shows an assembly view of the assembly of FIG. 15;

FIG. 17 shows an exploded view of another example assembly according to aspects of the present disclosure;

FIG. 18 shows perspective view of the assembly of FIG. 17;

FIG. 19 shows the outer surface of the assembly of FIG. 17;

FIG. 20 shows the inner surface of the assembly of FIG. 17;

FIG. 21 shows a simplified exploded view of the assembly of FIG. 17;

FIG. 22 shows an example wearable article according to aspects of the present disclosure;

FIG. 23 shows a detailed view of part of the wearable article in FIG. 22 in which the outer layer of material is transparent to show an electronics module positioned within the pocket space;

FIG. 24 a flow diagram for an example method of making an article according to aspects of the present disclosure;

FIG. 25 shows an example system according to aspects of the present disclosure;

FIG. 26 shows a schematic diagram for an example electronics module according to aspects of the present disclosure;

FIG. 27 shows an exploded view of another example electronics module according to aspects of the present disclosure; and

FIG. 28 shows the bottom surface of the electronics module of FIG. 27.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise.

“Wearable article” as referred to throughout the present disclosure may refer to any form of article which may be worn by a user such as a smart watch, necklace, bracelet, or glasses.

The wearable article may be a garment. The garment may refer to an item of clothing or apparel. The garment may be a top. The top may be a shirt, t-shirt, blouse, sweater, jacket/coat, or vest. The garment may be a dress, brassiere, shorts, pants, arm or leg sleeve, vest, jacket/coat, glove, armband, underwear, headband, hat/cap, collar, wristband, stocking, sock, or shoe, athletic clothing, personal protective equipment, swimwear, wetsuit or drysuit

The garment may be a tight-fitting garment. Beneficially, a tight-fitting garment helps ensure that the sensor devices of the garment are held in contact with or in the proximity of a skin surface of the wearer. The garment may be a compression garment. The garment may be an athletic garment such as an elastomeric athletic garment.

The wearable articles may be constructed from natural fibres, synthetic fibres, or a natural fibre blended with one or more other materials which can be natural or synthetic. The yarn may be cotton. The cotton may be blended with polyester and/or viscose and/or polyamide according to the particular application. Silk may also be used as the natural fibre. Cellulose, wool, hemp and jute are also natural fibres that may be used in the wearable article. Polyester, polycotton, nylon and viscose are synthetic fibres that may be used in the wearable article.

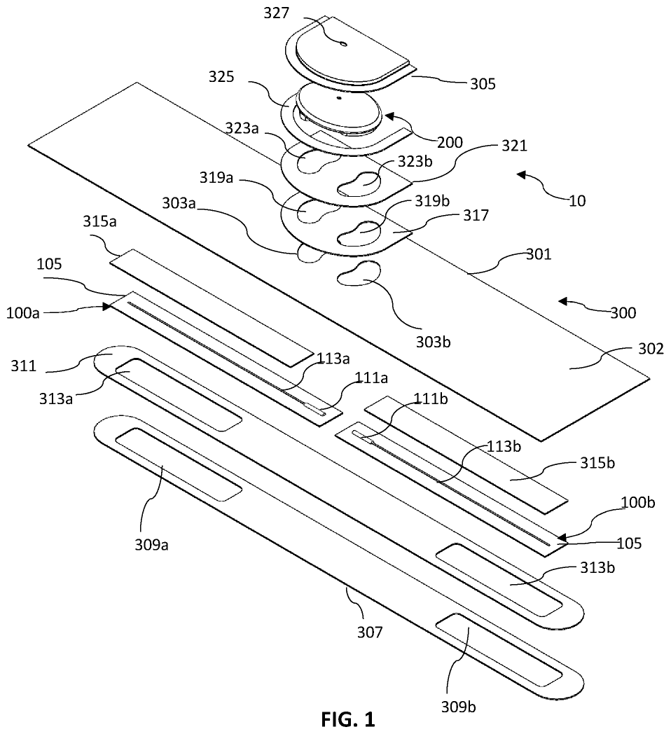

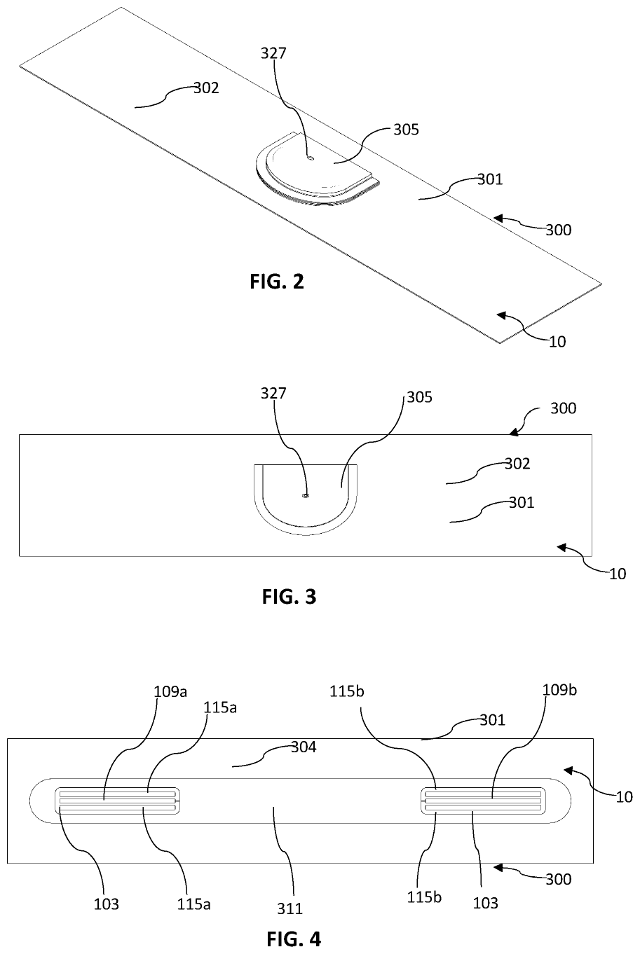

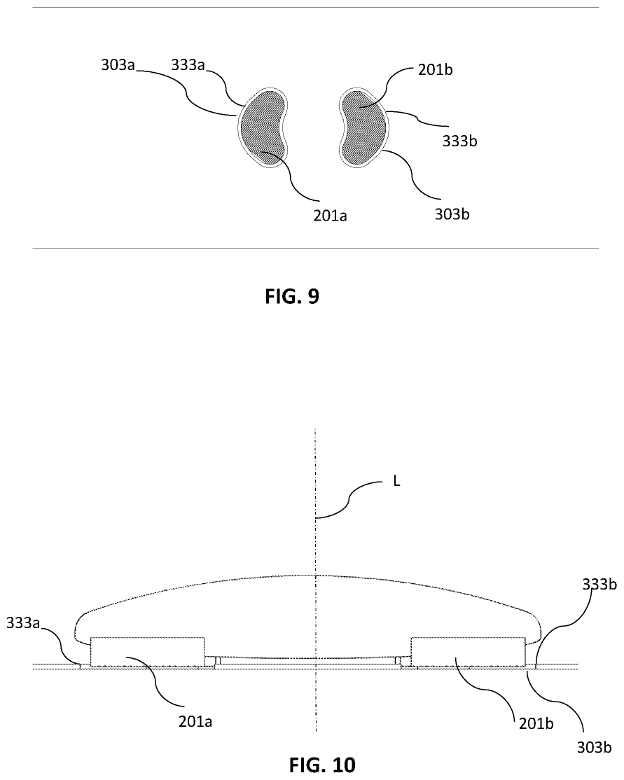

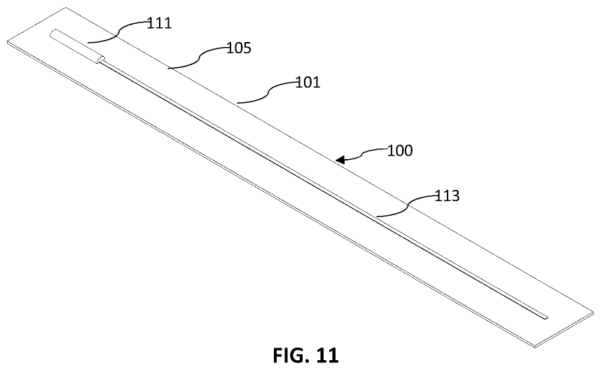

Referring to FIGS. 1 to 16, there is shown an assembly 10 according to aspects of the present disclosure. The wearable assembly 10 comprises an electronics module 200 and a wearable article 300.

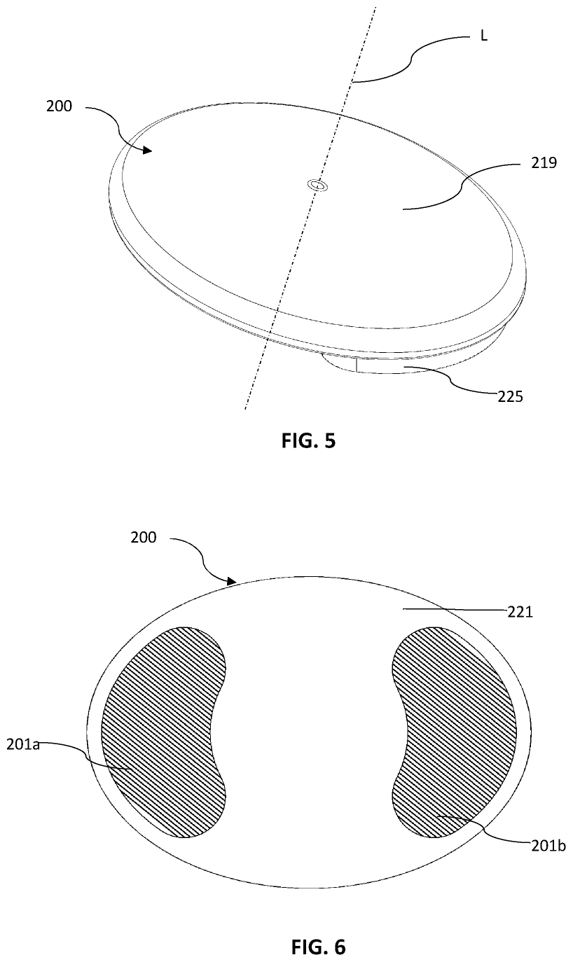

The electronics module 200 (FIGS. 5 and 6) comprises a housing 219, 221. The housing 219, 221 has a top surface 219 and a bottom surface 221. The electronics module 200 further comprises a plurality (two in this example) of interface elements 201a, 201b provided on an outside surface of the housing 219, 221. The interface elements 201a, 201b comprise conductive pads 201a, 201b provided on the bottom surface 221 of the housing 219, 221.

The wearable article 300 in this example forms a band of material that may extend around the circumference of the wearer of the article 300. It will be appreciated that only part of the wearable article 300 is shown in the Figures. The band may be a chest band, arm band, waist band or underband of a bra, for example.

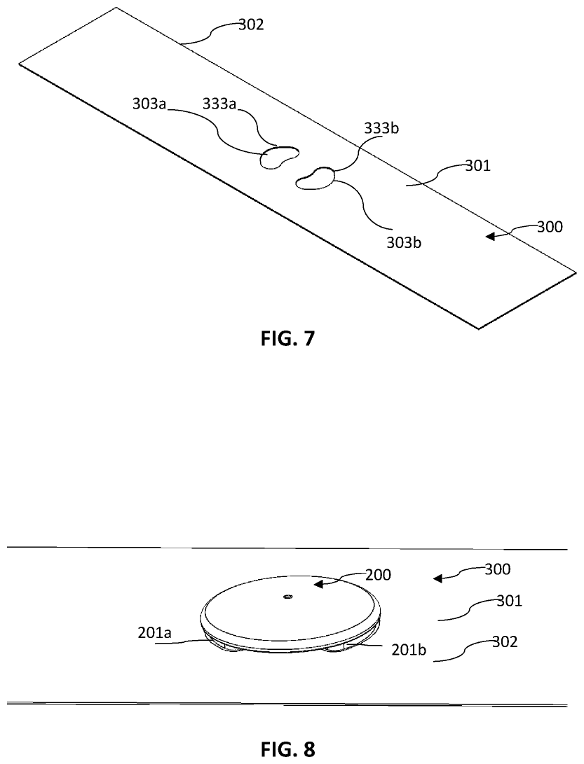

The wearable article 300 comprises a first layer of flexible material 301 (FIGS. 1 and 7). The first layer of material 301 is a fabric layer. The first layer of material 301 has an outer surface 302 that faces away from the wearer when the article 300 is worn, and an inner surface 304 that faces towards the skin surface of the wearer when the article 300 is worn. Two recesses 303a, 303b are provided in the first layer of material 301. The recesses 303a, 303b are openings 303a, 303b that extend through the first layer of material 301 from the outer surface 302 to the inner surface 304. The first layer of material 301 may form a band. That is the first layer of material 301 may form a loop of material that may extend around a circumference of the wearer. The first layer of material 301 may be a continuous strip of material such as a connected loop of material.

The electronics module 200 is seated on the outer surface 302 of the first layer of material 301 (FIG. 8). The electronics module 200 is seated such that the conductive pad 201a extends partially through recess 303a and conductive pad 201b extends partially through recess 303b.

The electronics module 200 is not permanently attached to the first layer of material 301 and may be removed and repositioned on the first layer of material 301 as desired.

The wearable article 300 comprises a plurality (two in this example) of sensing components 100a, 100b (FIGS. 1, 4 and 11 to 14) attached to the inner surface 304 of the first layer of material 301 by adhesive layers 315a, 315b. The sensing components 100a, 100b have an outer surface 105 that faces the inner surface 304 of the first layer of material 301 and an inner surface 103 that faces away from the inner surface 304 of the first layer of material 301. The inner surface 103 of the sensing components 100a, 100b faces towards the skin surface of the wearer when the article 300 is worn.

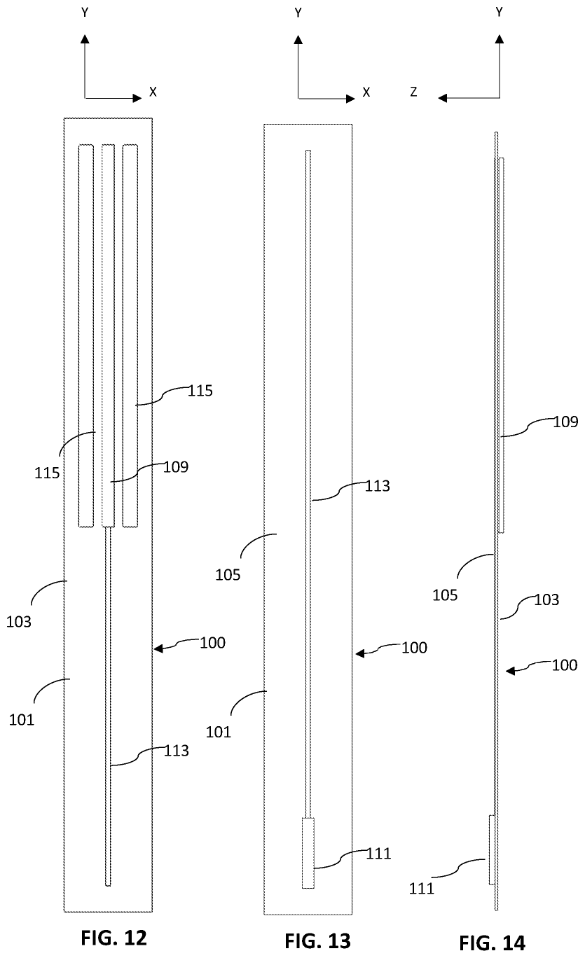

The sensing components 100, 100a, 100b comprise a base component 101. The base component 101 is a non-conductive fabric layer. The base component 101 may be knitted or woven from non-conductive yarn. The base component 101 has an inner surface 103 and an outer surface 105 opposing the first surface 103. The inner surface 103 and the outer surface 105 are parallel to one another and spaced apart along the Z axis (FIGS. 12 to 14).

The sensing components 100, 100a, 100b further comprise conductive regions 109, 109a, 109b, 111, 111a, 111b, 113, 113a, 113b formed of conductive yarn which is integrally knit or woven with the base component 101 to form a sensing component 100, 100a, 100b of an integral construction. That is the sensing component 100, 100a, 100b is formed from a continuous body of fabric. In this example, Circuitex™ conductive yarn from Noble Biomaterials Limited is used to form the conductive regions. Of course, other conductive yarns may be used. The conductive yarn may comprise a stainless steel yarn or a non-conductive or less conductive base yarn which is coated or embedded with conductive material such as carbon, copper and silver.

The sensing component 100, 100a, 100b comprises a first conductive region 109, 109a, 109b provided on the first surface 103 of the base component 101. The first conductive region 109, 109a, 109b is formed by knitting or weaving conductive yarn onto the first surface 103 to form a raised section of conductive material 109, 109a, 109b that extends away from the first surface 103 along the Z axis. This raised section of conductive material 109, 109a, 109b forms a raised electrode 109, 109a, 109b for contacting the skin surface of the wearer to measure signals from the wearer and/or introduce signals into the wearer. Having a raised electrode 109, 109a, 109b is beneficial in improving electrode contact with the skin surface particularly when the wearer is moving.

The electrode 109, 109a, 109b may be arranged to measure one or more biosignals of a user wearing the article 300. Here, “biosignal” may refer to any signal in a living being that can be measured and monitored. The electrode 109, 109a, 109b is generally for performing bioelectrical or bioimpedance measurements. Bioelectrical measurements include electrocardiograms (ECG), electrogastrograms (EGG), electroencephalograms (EEG), and electromyography (EMG). Bioimpedance measurements include plethysmography (e.g., for respiration), body composition (e.g., hydration, fat, etc.), and electroimpedance tomography (EIT). The electrode 109, 109a, 109b may additionally or separately be used to apply an electrical signal to the wearer. This may be used in medical treatment or therapy applications.

The sensing component 100, 100a, 100b further comprises a second conductive region 111, 111a, 111b provided on the second surface 105 of the base component 101. The second conductive region 111, 111a, 111b forms a connection terminal 111, 111a, 111b for electrically connecting with an electronics module 200. The second conductive region 111, 111a, 111b is formed by knitting or weaving conductive yarn onto the second surface 105 to form a raised conductive region 111, 111a, 111b that extends away from the second surface 105 along the Z axis. Having a raised connection terminal 111, 111a, 111b is beneficial in terms of improving the electrical connection between the connection terminal 111, 111a, 111b and the electronics module 200.

The connection terminal 111a is aligned with the opening 303a in the first layer of material 301 and extends partially into the opening 303a in the first layer of material 301. The connection terminals 111b is aligned with the opening 303b in the first layer of material 301 and extends partially into the opening 303b in the first layer of material 301. In some examples, the connection terminals 111a, 111b extend through the openings 303a, 303b such that sections of the connection terminals 111a, 111b are located on the outer surface 302.

The sensing component 100, 100a, 100b further comprises a conductive pathway 113, 113a, 113b of conductive material extending from the raised electrode 109a, 109b to the connection terminal 111, 111a, 111b. The conductive pathway 113, 113a, 113b electrically connects the raised electrode 109, 109a, 109b to the connection terminal 111, 111a, 111b. The conductive pathway 113, 113a, 113b is formed of conductive yarn which is knitted or woven into the first surface 103 of the base component 101.

The conductive pathway 113, 113a, 113b is knitted or woven along the length (in the Y axis direction) of one or both of the first surface 103 and the second surface 105 from the raised electrode 109, 109a, 109b to the connection terminal 111, 111a, 111b. The conductive pathway 113, 113a, 113b is incorporated into the base component 101 and is thus flush with the base component 101. In some examples, the conductive pathway 113, 113a, 113b extends along the inner or outer surface 103, 105. Having a conductive pathway 113, 113a, 113b which is flush with or minimally extends from a surface 103, 105 of the base component 101 is beneficial in terms of improving comfort and minimising the visual appearance of the sensing component 100, 100a, 100b on the wearable article 300. This is particularly important when the third layer of material 307 (FIGS. 1 and 4) is applied to the sensing component 100, 100a, 100b to insulate the conductive pathway 113a, 113b and prevent the conductive pathway 113a, 113b from forming a conductive connection with the skin surface of the wearer when worn. If the conductive pathway 113, 113a, 113b is too thick, then the insulating bonding layer may protrude above the electrode 109, 109a, 109b and push the electrode 109, 109a, 109b away from the skin surface.

The electrode 109, 109a, 109b is wider along the X axis than the conductive pathway 113, 113a, 113b. Having a wider electrode 109, 109a, 109b is beneficial in providing increased surface area of electrode contact with the skin surface S. Having a narrower conductive pathway 113, 113a, 113b is beneficial in terms of improving comfort for the wearer and minimising the visual appearance of the sensing component 100, 100a, 100b on the fabric article 100. The connection terminal 111, 111a, 111b is also wider along the X axis than the conductive pathway 113, 113a, 113b. Having a wider connection terminal 111, 111a, 111b is beneficial in terms of improving the electrical connection between the connection terminal 111, 111a, 111b and the electronics module 200.

The present disclosure is not limited to any particular dimension of the electrode 109, 109a, 109b, conductive pathway 113, 113a, 113b, and connection terminal 111, 111a, 111b.

Generally, however, the electrode 109, 109a, 109b, the conductive pathway 113, 113a, 113b, and connection terminal 111, 111a, 111b extend for a height of between 0.2 mm and 30 mm along the Z-axis.

The electrode 109a, 109b, conductive pathway 113a, 113b, and connection terminal 111a, 111b extend for a width of at least 0.1 mm along the X axis. The electrode 109a, 109b and/or connection terminal 111a, 111b may extend for a width of at least 0.5 mm, at least 1 mm, at least 2 mm, or at least 3 mm. The electrode 109a, 109b and/or connection terminal 111a, 111b may have a width of at least 3 mm, at least 5 mm, at least 10 mm, at least 15 mm, at least 20 mm, or at least 50 mm. The electrode 109a, 109b and/or connection terminal 111a, 111b may have a width between 5 mm and 20 mm.

The electrode 109a, 109b, conductive pathway 113a, 113b, and connection terminal 111a, 111b extend for a length of at least 1 mm along the Y axis. The electrode 109a, 109b may have a length of at least 5 mm, at least 10 mm, at least 20 mm, at least 50 mm, or at least 100 mm. The electrode 109a, 109b may have a length of between 20 and 50 mm. The connection terminal 111a, 111b may have a length of at least 5 mm, at least 10 mm, at least 20 mm, at least 50 mm, or at least 100 mm. The connection terminal 111a, 111b may have a length of between 5 mmm and 10 mm. The conductive pathway 113a, 113b may extend for a least of at least 5 mm, at least 10 mm, at least 20 mm, at least 50 mm, at least 100 mm, at least 200 mm, at least 300 mm, at least 500 mm. The conductive pathway 113a, 113b may extend for a length of the between 100 mm and 300 mm.

The sensing component 100, 100a, 100b further comprises a gripper component 115, 115a, 115b provided on the first surface 103 of the base component 101. The gripper component 115, 115a, 115b is arranged to grip the sensing component 100, 100a, 100b to the skin surface and hold it in place even when the wearer is moving.

The gripper component 115, 115a, 115b comprises two strips of gripper material provided along opposed edges of the electrode 109, 109a, 109b. The gripper component 115, 115a, 115b is provided around the periphery of the electrodes 109, 109a, 109b. Having the gripper material 115, 115a, 115b around both sides of the electrodes 109, 109a, 109b is beneficial in terms of enhancing the gripping effect, and also provides a barrier around the electrodes 109, 109a, 109b to help reduce water ingress and/or egress.

The gripper component 115, 115 is not provided around the periphery of the conductive pathway 113a, 113b in this example but this can be provided if desired. Moreover, the gripper component 115, 115 is only provided on the first surface 103 of the base component 101 and is not provided on the second surface 105 of the base component 101. In some examples, it may also be desirable to provide gripper component 115a, 115b on the second surface 105.

The gripper component 115, 115a, 115b is a raised section that extends away from the first surface 103 of the base component 101. This means that the gripper component 115, 115a, 115b has a raised or three-dimensional profile which helps place it in contact with the skin surface without requiring additional compression from the base component 101. The electrodes 109, 109a, 109b still extend from the first surface 103 to a greater extent than the gripper component 115, 115a, 115b.

In one example, the gripper component 115, 115a, 115b is formed by applying silicone tape to either side of the electrodes 109, 109a,109b on the first surface 103. Before applying the tape to the first surface 103 of the base component 101, the desired application areas for the gripper component 115, 115a, 115b may be marked on the first surface 103 using, for example, a particular colour. This may help a person or machine to apply the silicone tape. An example silicone tape is the Stay4Sure™ tape as provided by Stretchline Holdings 1430 Broadway Suite 307, New York, N.Y. 10018 U.S.A.

In another example, the gripper component 115, 115a, 115b is formed by applying a coating of silicone to the first surface 103 of the base component 101. This may be formed by applying a bead of silicone material to the first surface 103 of the base component 101.

In another example, the gripper component 115, 115a, 115b is formed by knitting/weaving silicone yarn (or other yarn having similar gripping/non-slip properties to silicone yarn) after or during the process of manufacturing the rest of the sensing component 100, 100a, 100b. This approach can simplify construction as additional steps such as the application of a silicone tape or a silicone coating are not required. An example silicone yarn is Silicotex® as provided by Massebeuf Textiles, 135 route de la Fabrique, 07380 Pont de Labeaume.

In some examples, particularly when the gripper component 115, 115a, 115b is formed by knitting/weaving silicone yarn, the fabric article may further comprise filler material disposed within the gripper component 115, 115a, 115b. The filler material raises the profile of the gripper component 115, 115a, 115b away from the base component 101. This helps to increase the quality, consistency and area of contact. The filler material comprises an expanding yarn that is knitted or woven into the fabric article during the process of knitting/weaving the fabric article 100. The expanding yarn used in this example is a Newlife™ polyester filament yarn manufactured by Sinterama S.p.A.

Beneficially, the filler material raises the profile of the gripper component 115, 115 away from the base component 101. This helps to increase the quality, consistency and area of contact area. This is particularly beneficial as it helps ensure contact against the skin surface S without requiring additional compression such as through additional elastomeric material. The filler material maintains the shape of the gripper component 11a, 115 and protects against deformation, buckle and roll even when they are rubbed against the skin or other surface. Moreover, using an expanding yarn means that the process of filling out the gripper component 115, 115 is an intrinsic part of the manufacturing process. A separate manual process of inserting filler material into already formed gripper component 115, 115 is not required.

Referring to FIGS. 1, 8, 9, 10, 15 and 16, when the electronics module 200 is seated on the first layer of material 301, the conductive pads 201a, 201b of the electronics module 200 extend into the openings 303a, 303b and contact the connection terminals 111a, 111b of the sensing components 100a, 100b. In particular, the conductive pad 201a extends into the opening 303a to contact the connection terminal 111a and the conductive pad 201b extends into the opening 303b to contact the connection terminal 111b. In this way, the electronics module 200 is electrically connected to the sensing components 100a, 100b and is able to receive signals from the electrodes 109a, 109b.

The openings 303a, 303b are sized to restrict movement of the electronics module 200 along the outer surface 302 of the first layer of material 301. This means that the translational movement of the electronics module 200 along the surface 302 of the first layer of material 301 is restricted by the openings 303a, 303b. Further, the openings 303a, 303b are sized to restrict axial movement of the electronics module 200 about the longitudinal axis L of the electronics module 200. This means that the openings 303a, 303b restrict rotational movement of the electronics module 200 about the longitudinal axis L. The longitudinal axis L is perpendicular to the outer surface 302 of the first layer of material 301 when the electronics module 200 is seated on the first layer of material 301.

Restricting movement of the electronics module 200 along the outer surface 302 of the first layer of material 301 is beneficial as it prevents the conductive pads 201a, 201b from sliding relative to the connection terminals 111a, 111b of the sensing components 100a, 100b. This sliding of the conductive pads 201a, 201b could cause the conductive pads 201a, 201b to move out of electrical contact with the connection terminals 111a, 111b or cause one or both of the conductive pads 201a, 201b to be connected to both connection terminals 111a, 111b at the same time which could form an electrical short. Restricting axial movement of the electronics module 200 about the longitudinal axis L of the electronics module 200 is beneficial for the same reasons. Therefore, the openings 303a, 303b are able to prevent translational and/or axial motion of the electronics module 200 which may normally be caused in wearable articles as a result of motion of the wearer. Motion of the wearer is a particular problem in terms of providing and maintaining electrical contact in wearable articles used in sports and fitness.

In addition, the openings 303a, 303b provide a locating mechanism that facilitates insertion of the electronics module 200 correctly into the pocket space to form the electrical connection. This makes it easier for a user to correctly insert the electronics module 200 into the pocket space.

The openings 303a, 303b are bounded by edges 333a, 333b (FIGS. 7, 8 and 9) of the first layer of material 301. This means that edges of the first layer of flexible material form the walls of the openings 303a, 303b. The openings 303a, 303b are not bounded by a rigid material such as a metal. The first layer of material 301 that bounds the edges 333a, 333b may be a non-conductive material. The edges 333a, 333b form barriers 333a, 333b that abut against the parts of the conductive pads 201a, 201b disposed within the openings 303a, 303b. In this way, when the electronics module 200 attempts to move along the surface of the first layer of material 301, the conductive pads 201a, 201b move into or move into further abutment with barriers 333a 333b. The abutment between the conductive pads 201a, 201b and the barriers 333a, 333b means that further movement of the electronics module 200 along the surface 302 of the first layer of material 301 is restricted.

Two openings 303a, 303b are provided to correspond to the two conductive pads 201a, 201b of the electronics module 205. The edges 333a, 333b of the openings 303a, 303b have profiles which correspond to the profiles of the conductive pads 201a, 201b. In particular, the edges 333a, 333b have arcuate profiles which correspond to the arcuate profiles of the conductive pads 201a, 201b.

The openings 303a, 303b in this example are formed to have complementary shapes to the cross-sectional shape of the conductive pads 201a, 201b of the electronics module 200. Both the openings 303a, 303b and the conductive pads 201a, 201b have kidney bean shapes in this example.

The openings 303a, 303b are sized to be slightly larger than the cross-sectional shape of the conductive pads 201a, 201b to allow for the conductive pads 201a, 201b to extend into the openings 303a, 303b, but provide a sufficiently tight fit to restrict movement of the electronics module 200 along the surface 302 of the first layer of material 301. In some examples, the openings 303a, 303b may be between 0.1 mm and 2 mm larger than the conductive pads 201a, 201b optionally between 0.5 mm and 2 mm, optionally between 1 mm and 2 mm. The openings 303a, 303b may also be more than 2 mm larger than the conductive pads 201a, 201b in some examples.

The openings 303a, 303b are not required to have the same shape as the conductive pads 201a, 201 b to achieve the effect of restricting translational/axial movement. Moreover, the conductive pads 201a, 201b and openings 303a, 303b are not required to have cross-sections in the form of kidney bean shapes as shown in FIGS. 6 and 10. Other shapes such as square, rectangular, oval and circular may be used, or any other polygon chosen as appropriate by the skilled person may be used.

The wearable article 300 comprises a second layer of material 305 (FIGS. 1 to 3) that is positioned adjacent to and external to the first layer of material 301. The second layer of material 305 is attached to the first layer of material 301 to form a pocket space sized to receive and contain the electronics module 200. The second layer of material 305 is attached to the first layer of material 301 via intermediate layers 317, 321, 325. The side and lower margins of the second layer of material 305 are attached to the first layer of material 301 while the upper edge of second layer of material 305 is unaffixed to the first layer of material 301. The upper edge of the second layer of material 305 forms an opening to enable the pocket space to be accessed from outside the wearable article 300. The second layer of material 305 comprises an opening or window 327 to enable a light source of the electronics module 200 to be visible externally.

The second layer of material 305 acts as an attachment mechanism that applies pressure to the electronics module 200 to urge the electronics module 200 towards the surface 302 of the first layer of material 301. In particular, the second layer of material 305 comprises an elastomeric material that applies pressure to the electronics module 200. Beneficially, the second layer of material 305 helps restrict movement of the electronics module 200 in the direction of the longitudinal axis L. This helps prevent the conductive pads 201a, 201b from moving out of contact with the connection terminals 111a, 111b. Thus, in this example, the combination of the openings 303a, 303b and the second layer of material 305 means that the wearable article 300 restricts movement of the electronics module 200 in the direction of the outer surface 302, rotational movement of the electronics module 200 about the longitudinal axis L, and movement of the electronics module 200 along the longitudinal axis L. In this way, the present disclosure is able to maintain the electrical connection between the connection terminals 111a, 111b and the conductive pads 201a, 201b even under vigorous motion of the wearer of the assembly 10 all without requiring a permanent mechanical attachment between the electronics module 200 and the article 300.

The intermediate layers 317, 321, 325 comprise adhesive layers 317, 325 and a waterproof layer 319 sandwiched between the interface layers 317, 325. The waterproof layer 319 is formed from a waterproof film of material.

The conductive pads 201a, 201b of the electronics module 200 are arranged to extend through openings 323a, 323b in the waterproof layer 321, openings 319a, 319b in the adhesive layer 317, and into the openings 303a, 303b in the first layer of material 301 so as to electrically connect with the connection terminals 111a, 111b of the sensing components 100a, 100b. The openings 319a, 319b, 323a, 323b, are aligned with and have shapes that correspond to the shapes of the opening 303a, 303b.

It will be appreciated that electronics module 200 is still seated on the outside surface 302 of the first layer of material 301 even though the layers 317, 321 are disposed between the outer surface 302 and the electronics module 200. That is, seating the electronics module 200 on the outer surface 302 does not require the electronics module 200 to be in direct contact with the outer surface 302.

The openings 323a, 323b in the waterproof layer 321 are sized such that, when the conductive pads 201a, 201b are positioned in the openings 323a, 323b, ingress of water through the recess is restricted. The openings 323a, 323b are similarly constructed to the openings 303a, 303b in the first layer of material 301 and in this example have a corresponding size and shape to the openings 303a, 303b in the first layer of material 301. The openings 323a, 323b may also help to restrict translational and rotational movement of the electronics module 200 in the same way as the openings 303a, 303b.

Advantageously, the waterproof layer 321 prevents against water ingress into the pocket space when the electronics module 200 is disposed in the pocket space and the conductive pads 201a, 201b are positioned in the openings 323a, 323b. Because the conductive pads 201a, 201b form a tight fit with the openings 323a, 323b water ingress through the openings 323a, 323b is restricted. Moreover, the waterproof layer 321 which in this example is slightly larger than the pocket space prevents water ingress through the potentially water permeable first layer of material 301. As a result, moisture such as sweat from the wearer is restricted from entering the pocket. This helps protect the electronics module 200 against damage due to water ingress. In addition, this helps protect against water in the pocket space forming an electrical short between the conductive pads 201a, 201b as a result of water ingress.

The wearable article 300 comprises a third layer of material 307 (FIGS. 1, 4, 15 and 16) that is attached to the inner surface 103 of the sensing components 100a, 100b by adhesive layer 311. The third layer of material 307 and the adhesive layer 311 comprise openings 309a, 309b, 313a, 313b. The openings 309a, 309b, 313a, 313b are aligned with the first conductive regions 109a, 109b on the inner surface 103 of the sensing components 100a, 100b. This means that the first conductive regions 109a, 109b are not covered by the third layer of material 307.

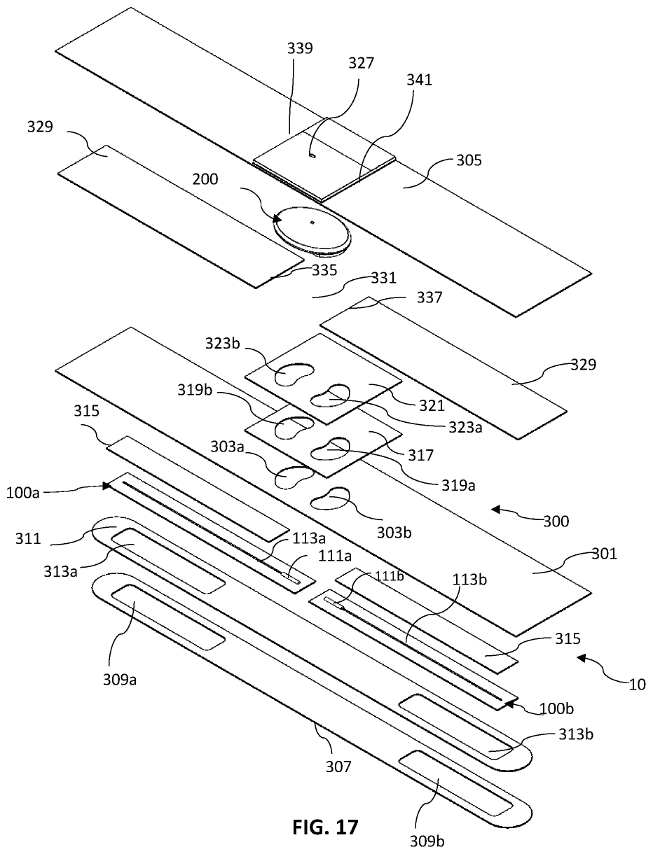

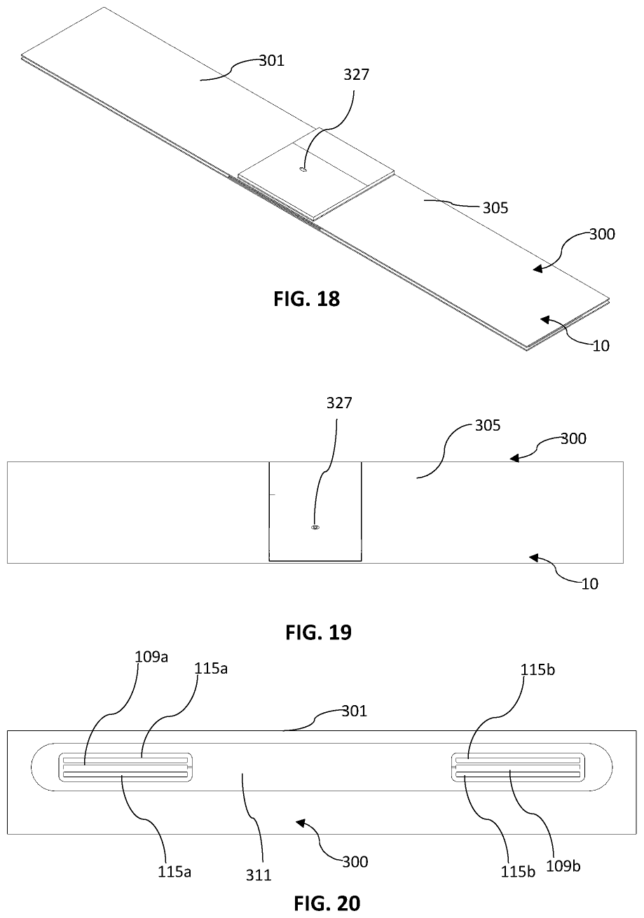

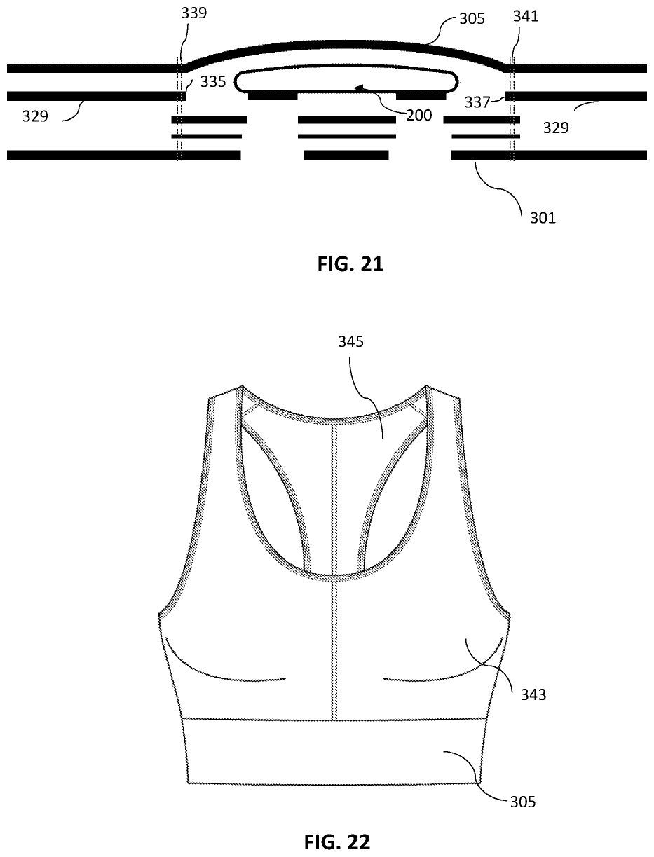

Referring to FIGS. 17 to 23 there is shown another example wearable assembly 10 according to aspects of the present disclosure. The wearable assembly 10 is similar to the wearable assembly 10 of FIGS. 1 to 16 and like reference numerals are used to indicate like components.

The second layer of material 305 is a longer strip of material that has similar dimensions to the first layer of material 301. In addition, an elastomeric material 329 is provided between the first and second layers of material 301, 305. The elastomeric material 329 is arranged to tension the wearable article 300 when worn.

The elastomeric material 329 defines an open region 331 in the pocket space formed between the first and second layers 301, 305 of material. In this way, the elastomeric material 329 does not affect the communication between the electronics module 200 and the connection terminals 111a, 111b when the electronics module 200 is positioned in the pocket space.

The elastomeric material 329 is a continuous strip of material that comprises a first end 335 and a second end 337. The first end 335 and the second end 337 are spaced apart from one another to define the open region 331. That is, the first end 335 and the second end 337 of the elastomeric material 329 are not connected to one another.

The second layer 305 is stitched to the first layer 301 along the lines 339, 341 either side of the pocket space. The stitches along the line 339 extend through the first end 335 of the elastomeric material 329 to join the first end 335 to the first and second layers of material 301, 305. The stitches along the line 341 extend through the second end 337 of the elastomeric material 329 to join the second end 337 to the first and second layers of material 301. Therefore, in this example, rather than joining the first end 335 and second end 337 of the elastomeric material 329 together to form a continuous loop of material, the ends 335 and 337 are not connected to one another and are spaced apart to define the open region 301. The attachment of the second layer of material 305 to the first layer of material 301 to form the pocket space joins the elastomeric layer 329 to the first and second layers of material 301, 305. The elastomeric material 329 may only be connected to the first and second layers of material 301, 305 along the lines 339, 341.

The first layer 301, second layer 305 and elastomeric material 329 form a band of material which, when worn extends around the circumference of at least part of the wearer of the wearable article 300.

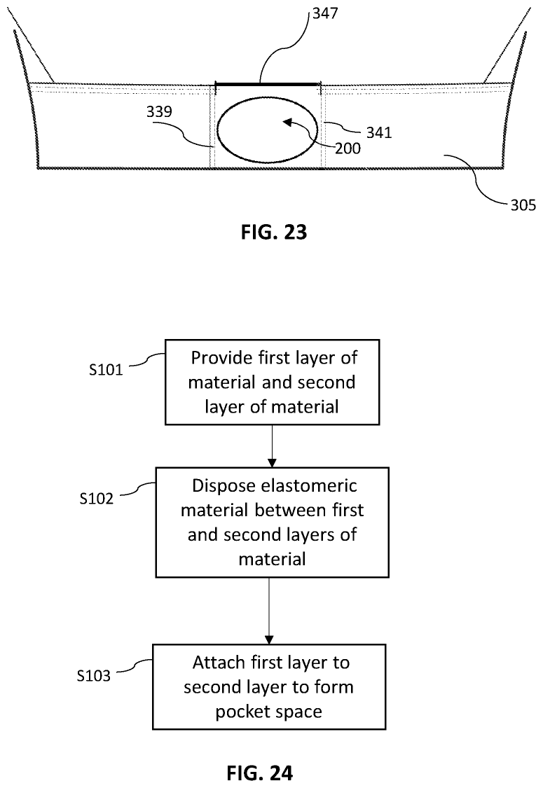

FIGS. 22 and 23 show an example of the wearable article 300 that is in a form of a bra comprising a front portion 343 and back portion 345. The band formed by the first layer 301, second layer 305 and elastomeric material 329 is an underband of the bra that extends from a lower margin of the front portion and the back portion of the bra. The underband surrounds the circumference of the wearer of the wearable article 300. The pocket space is formed in a central front region of the bra as shown in FIG. 23. The pocket opening 347 is provided at the top of the underband to allow for the electronics module 200 to be inserted into and removed from the pocket space.

Referring to FIG. 24, there is shown an example method of manufacturing a wearable article according to aspects of the present disclosure. Step S101 of the method comprises providing a first layer of material and a second layer of material. Step S102 of the method comprises disposing an elastomeric material between the first and second layers of material, wherein the elastomeric material is arranged to tension the wearable article when worn. Step S103 of the method comprises attaching the second layer of material to the first layer of material to form a pocket space for receiving an electronics module, wherein the elastomeric material defines an open region in the pocket space for receiving the electronics module.

While the above examples refer generally to sensing components 100, 100a, 100b formed using knitting and weaving techniques the present disclosure is not limited to these examples. The sensing components 100, 100a, 100b can comprise any desired conductive material and are not limited to knitted and woven conductive yarns. The conductive material may include printed conductive ink or conductive transfers formed from layers of insulating and conductive ink. Other forms of conductive material that can be incorporated onto a fabric are within the scope of the present disclosure. The sensing components 100, 100a, 100b in some examples may be integral with the first layer of material 301.

It will be appreciated that some or all of the adhesive layers 317, 325, 311, 315a, 315b may not be required in all examples of the wearable article 300. Some components may have integral adhesive meaning that separate adhesive layers are not required. A different method of joining components together such as through stitching may be provided so that an adhesive is not required. In some examples, components may be integrally formed with one another such that an adhesive is not necessary. For example, the second layer of material 305 may be integrally formed with the first layer of material 301. That is the second layer of material 305 and the first layer of material 301 may be a single piece of material that is folded over to form the pocket space.

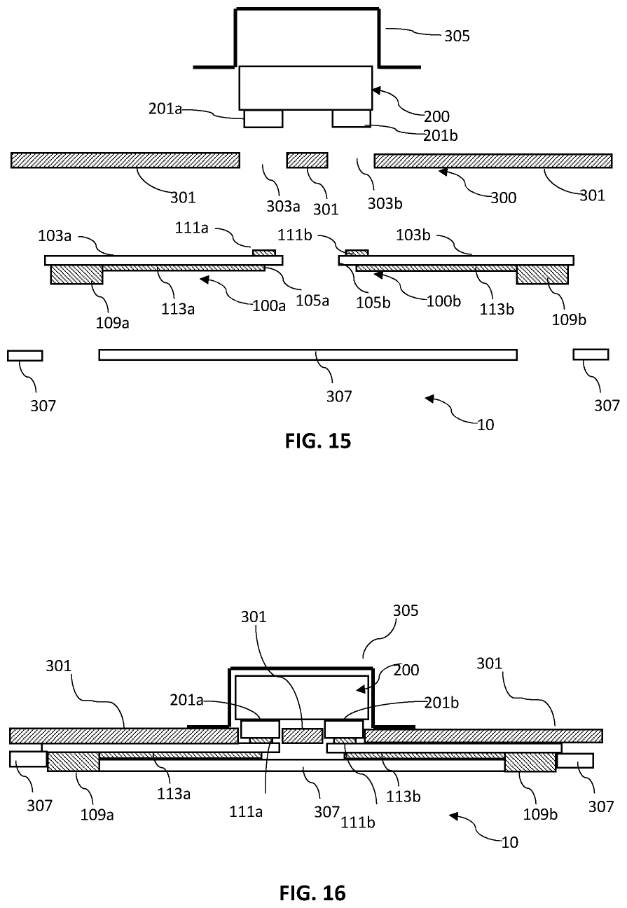

It will be appreciated that the waterproof layer 321 is not required in all examples of the wearable article 300. The first layer of material 301 may be waterproof at least in the section adjacent to the pocket space. In addition, the electronics module 200 may be constructed in a way that avoids the need for separate waterproofing. FIGS. 15 and 16 show a simplified example of the wearable assembly 10 where the layers 317, 325, 311, 315a, 315b and 321 are omitted.

It will be appreciated that the third layer of material 307 is not required in all examples of the present disclosure and may be omitted depending on application. For example, the sensing component 100a, 100b may include shielding for the conductive pathway 113a, 113b.

It will be appreciated that in some examples, that the electronics module 200 is not required to contact the connection terminals 111a, 111b to communicate with the sensing component 100a, 100b. For example, the sensing component 100a, 100b and the electronics module 200 may wirelessly communicate by forming an inductive coupling. The sensing component 100a, 100b and the electronics module 200 may both comprise an antenna for forming the inductive coupling. In this example providing recesses 303a, 303b in the first layer 301 and/or recesses 323a, 323b the waterproof layer 321 is still beneficial in terms of restricting movement of the electronics module 200 so as to form and maintain the inductive coupling. In these examples, the interface element 201 of the electronics module 200 is not required to be conductive and may be a non-conductive locating mechanism although it still may be in the form of one or more pads.

Moreover, providing the second layer 305 to urge the electronics module 200 towards the first layer of material 301 is still beneficial in terms of reducing the communication distance between the electronics module 200 and the connection terminal 111a, 111b. However, in this example, the recesses 303a, 303b and 323a, 323b are not required to be openings that extend through the first layer of material 301 and/or the waterproof layer 321. The recesses 303a, 303b 323a, 323b may only extend part of the way through the first layer of material and/or the waterproof layer 321. The recesses 323a, 323b may only be provided in the waterproof layer 321 and not in the first layer 301.

It will be appreciated that two separate sensing components 100a, 100b are not required in all aspects of the present disclosure. The sensing components 100a, 100b may be connected together. A single sensing component may be provided comprising any number of connection terminals and electrodes or other sensing circuitry.

It will be appreciated that the number of recesses 303a, 303b is not required to correspond to the number of interface elements 201a, 201b but this is generally preferred. The electronics module 200 may comprise a plurality of interface elements 201a, 201b and the first layer of material 301 may comprise a single recess for receiving the plurality of interface elements. The first layer of material 301 may comprise more than one recess but the number of recesses may be less than the number of interface elements 201a, 201b.

It will be appreciated that the attachment mechanism does not need to be formed by the second layer of material 305. The second layer of material 305 is not required in all aspects of the present disclosure. The second layer of material 305 may comprise a magnet arranged to cooperate with a magnet mounted within the electronics module 200 to releasably attach the electronics module 200 to the wearable article 300 and restrict movement in the direction of the longitudinal axis L.

It will also be appreciated that the interface elements 201a, 201b are not required to extend into the openings 303a, 303b in all examples. Instead, the connections between the connection terminals 111a, 111b and the interface elements 201a, 201b may be provided on the outer surface if the connection terminals 111a, 111b extend through the openings 303a, 303b.

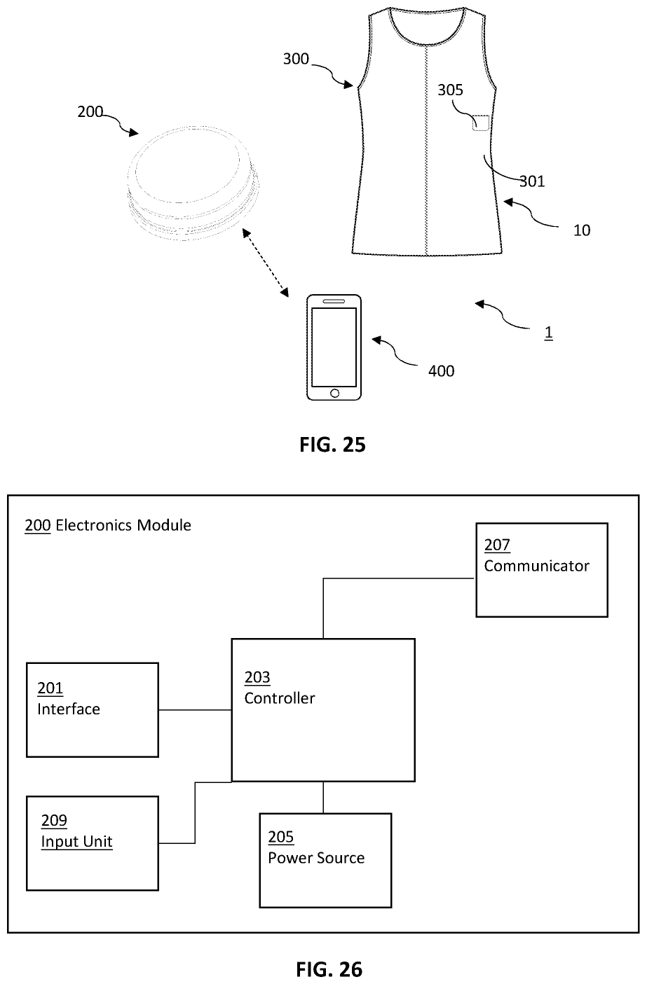

Referring to FIG. 25, there is shown an example system 1 according to aspects of the present disclosure. The system 1 comprises wearable assembly 10 and a mobile device 400.

The wearable assembly 10 comprises a wearable article 300 comprising first and second layers of material 301, 305 such as per the examples described above. The wearable article 300 in this example is a top 300. The first layer of material 301 is the base layer of the top 300. The second layer of material 305 is the outer layer of the pocket.

The electronics module 200 is able to be disposed within the pocket space formed by the first and second layers of material 301, 305. When positioned within the pocket space, the electronics module 200 is able to integrate with the sensing components so as to obtain signals from the sensing components. The electronics module 200 is further arranged to wirelessly communicate data to the mobile device 400. Various protocols enable wireless communication between the electronics module 200 and the mobile device 400. Example communication protocols include Bluetooth®, Bluetooth® Low Energy, and near-field communication (NFC).

The present disclosure is not limited to electronics modules 200 that communicate with mobile devices 400 and instead may communicate with any electronic device capable of communicating directly with the electronics module 200 or indirectly via a server over a wired or wireless communication network. The electronic device may be a wireless device or a wired device. The wireless/wired device may be a mobile phone, tablet computer, gaming system, MP3 player, point-of-sale device, or wearable device such as a smart watch. A wireless device is intended to encompass any compatible mobile technology computing device that connects to a wireless communication network, such as mobile phones, mobile equipment, mobile stations, user equipment, cellular phones, smartphones, handsets or the like, wireless dongles or other mobile computing devices. The wireless communication network is intended to encompass any type of wireless network such as mobile/cellular networks used to provide mobile phone services.

Beneficially, the removable electronic module 200 may contain all of the components required for data transmission and processing such that the wearable article 300 only comprises the sensing components. In this way, manufacture of the wearable article 300 may be simplified. In addition, it may be easier to clean a wearable article 300 which has fewer electronic components attached thereto or incorporated therein. Furthermore, the removable electronic module 200 may be easier to maintain and/or troubleshoot than embedded electronics. The electronic module 200 may comprise flexible electronics such as a flexible printed circuit (FPC). The electronic module 200 may be configured to be electrically coupled to the wearable article 300.

It may be desirable to avoid direct contact of the electronic module 200 with the wearer's skin while the wearable article 300 is being worn. It may be desirable to avoid the electronic module 200 coming into contact with sweat or moisture on the wearer's skin. The electronic module 200 may be provided with a waterproof coating or waterproof casing. For example, the electronic module 200 may be provided with a silicone casing.

Referring to FIG. 26, there is shown a schematic diagram of an example of the electronics module 200. The electronics module 200 comprises an interface 201, a controller 203, a power source 205, and a communicator 207.

The interface 201 is arranged to communicatively couple with the sensing component of the fabric article so as to receive a signal from the sensing component. The controller 203 is communicatively coupled to the interface 201 and is arranged to receive the signals from the interface 201. The interface 201 may form a conductive coupling or a wireless (e.g. inductive) communication coupling in some examples. That is, the connection terminal of the fabric article may be in the form of an antenna for inductively coupling to a corresponding antenna of the interface 201. The interface 201 may comprise conductive pads as described above.

The power source 205 is coupled to the controller 203 and is arranged to supply power to the controller 203. The power source 205 may comprise a plurality of power sources. The power source 105 may be a battery. The battery may be a rechargeable battery. The battery may be a rechargeable battery adapted to be charged wirelessly such as by inductive charging. The power source 205 may comprise an energy harvesting device. The energy harvesting device may be configured to generate electric power signals in response to kinetic events such as kinetic events performed by a wearer of the garment. The kinetic event could include walking, running, exercising or respiration of the wearer. The energy harvesting material may comprise a piezoelectric material which generates electricity in response to mechanical deformation of the converter. The energy harvesting device may harvest energy from body heat of a wearer of the garment. The energy harvesting device may be a thermoelectric energy harvesting device. The power source may be a super capacitor, or an energy cell.

The communicator 207 may be a mobile/cellular communicator operable to communicate the data wirelessly via one or more base stations. The communicator 207 may provide wireless communication capabilities for the wearable article and enables the wearable article to communicate via one or more wireless communication protocols such as used for communication over: a wireless wide area network (VWVAN), a wireless metroarea network (VVMAN), a wireless local area network (VVLAN), a wireless personal area network (VVPAN), Bluetooth® Low Energy, Bluetooth® Mesh, Bluetooth® 5, Thread, Zigbee, IEEE 802.15.4, Ant, a near field communication (NFC), a Global Navigation Satellite System (GNSS), a cellular communication network, or any other electromagnetic RF communication protocol. The cellular communication network may be a fourth generation (4G) LTE, LTE Advanced (LTE-A), LTE Cat-M1, LTE Cat-M2, NB-IoT, fifth generation (5G), sixth generation (6G), and/or any other present or future developed cellular wireless network. A plurality of communicators may be provided for communicating over a combination of different communication protocols.

The electronics module 200 may comprise a Universal Integrated Circuit Card (UICC) that enables the electronics module 200 to access services provided by a mobile network operator (MNO) or virtual mobile network operator (VMNO). The UICC may include at least a read-only memory (ROM) configured to store an MNO/VMNO profile that the electronics module 200 can utilize to register and interact with an MNO/VMNO. The UICC may be in the form of a Subscriber Identity Module (SIM) card. The electronics module 200 may have a receiving section arranged to receive the SIM card. In other examples, the UICC is embedded directly into the controller 203 of the electronics module 200. That is, the UICC may be an electronic/embedded UICC (eUICC). A eUICC is beneficial as it removes the need to store a number of MNO profiles, i.e.

electronic Subscriber Identity Modules (eSIMs). Moreover, eSIMs can be remotely provisioned to electronics modules 200. The electronics module 200 may comprise a secure element that represents an embedded Universal Integrated Circuit Card (eUICC).

The input unit 209 enables the electronics module 200 to receive a user input for controlling the operation of the electronics module 200. The input unit 209 may be any form of input unit capable of detecting an input event. The input event is typically an object being brought into proximity with the electronics module 200.

In some examples, the input unit 209 comprises a user interface element such as a button. The button may be a mechanical push button.

In some examples, the input unit 209 comprises an antenna. In these examples, the input event is detected by a current being induced in the first antenna. The mobile device 400 is powered to induce a magnetic field in an antenna of the mobile device 400. When the mobile device 400 is placed in the magnetic field of the antenna, the mobile device 400 induces current in the antenna.

In some examples, the input unit 209 comprises a sensor such as a proximity sensor or motion sensor. The sensor may be a motion sensor that is arranged to detect a displacement of the electronics module 200 caused by an object being brought into proximity with the electronics module 200. These displacements of the electronics module 200 may be caused by the object being tapped against the electronics module 200. Physical contact between the object and the electronics module 200 is not required as the electronics module 200 may be in a holder such as a pocket 305 of the garment 300. This means that there may be a fabric (or other material) barrier between the electronics module 200 and the object. In any event, the object being brought into contact with the fabric of the pocket will cause an impulse to be applied to the electronics module 200 which will be sensed by the sensor.

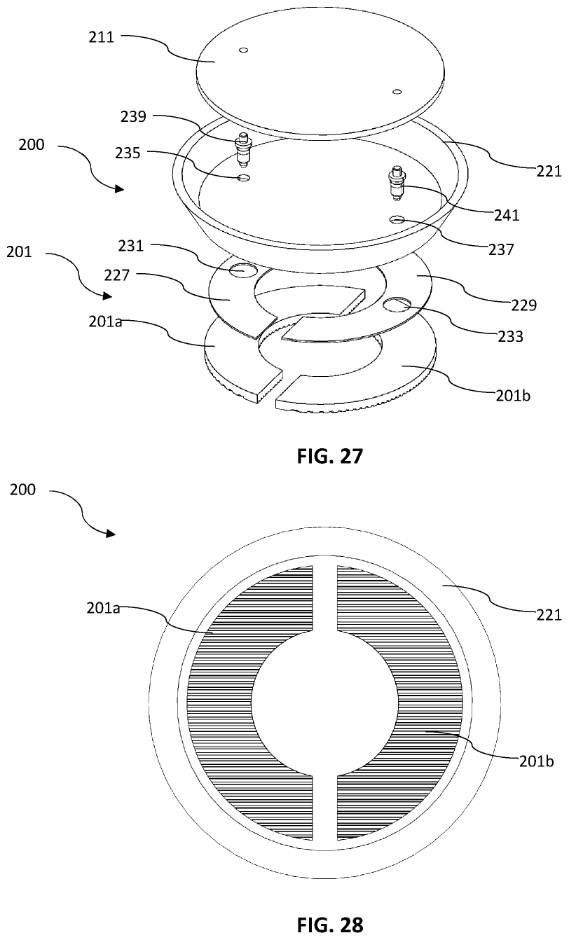

Referring to FIGS. 27 and 28, there is shown another example electronics module 200 according to aspects of the present disclosure. The top enclosure 219 is omitted in FIG. 27 so that the internal components of the electronics module 200 are visible. The top enclosure 219 is similar to the top enclosure 219 of FIG. 5.

Like the electronics module 200 of FIGS. 5 and 6, the electronics module comprises interface elements 201a, 201b in the form of conductive pads 201a, 201b. The two conductive pads 201a, 201b are adhesively attached to the external surface of the bottom enclosure 221 using adhesive layers 227, 229. The adhesive layers 227, 229 comprise openings 231, 233. These openings 231, 233 are aligned with openings 235, 237 provided in the bottom enclosure 221.

Pogo pins 239, 241 extend through openings 235, 237 in the bottom enclosure 221 and openings 231, 233 in the adhesive layers 227, 229 so as to electrically connect to the conductive pads 201a, 201b. The openings 231, 233 in the adhesive layers 227, 229 are larger than the openings 235, 237 in the bottom enclosure 221 to help ensure that adhesive does not interfere with the pogo pin mechanism or cause a potential short circuit. The pogo pins 239, 241 electrically connect the printed circuit board 211 to the conductive pads 201a, 201b.

Pogo pins 239, 241 are not required in all examples and other forms of force-biased conductor may be used.

The conductive pads 201a, 201b are formed from conductive elastomeric material 201a, 201b. The conductive elastomeric material used in this example is a conductive silicone rubber material, but other forms of conductive elastomeric material may be used. Beneficially, elastomeric material such as conductive silicone rubber can have an attractive visual appearance and may easily be moulded or extruded to have branded or other visual elements. The pads 201a, 201b may be textured to provide additional grip when positioned on the garment. The texture may be, for example, a ribbed or knurled texture. The elastomeric material 201a, 201b shown in FIGS. 27 and 28 has a ribbed texture. The conductive pads 201a, 201b are not required to be formed of elastomeric material other conductive materials such as metals or conductive fabric may be used.

The conductive pads 201a, 201b together form a split-ring shape, but other shapes and arrangements are within the scope of the present disclosure.

The housing 219, 221 has a circular cross-sectional shape in the example of FIGS. 27 and 28 but this is not required. The housing may have any cross-sectional shape such as oval, square or rectangular.

In summary there is provided an assembly 10, a wearable article 300 and a method of making the same. The assembly 10 comprises electronics module 200 and wearable article 300. Article 300 comprises a first layer of material 301 comprising a recess 303a arranged to receive an interface element 201a of the electronics module 200. The recess 303a restricts movement of the module 200 along the surface of the material 301, 321. The recess 303a may enable the interface element 201a to connect with a sensing element 100a attached to an underside surface of the material 301, 321. A second layer of material 305 may be attached to the material 301, 321 to define a pocket space for receiving the module 200. An elastomeric material 329 may be provided between the material layers 301, 321, 305 to tension the article 300 when worn. The elastomeric material 329 defines an open region in the pocket space for receiving the module 200. The first layer 301, 321 may be a waterproof layer 321.

In the present disclosure, the electronics module may also be referred to as an electronics device or unit. These terms may be used interchangeably.

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1-25. (canceled)

26. A wearable article comprising:
a first layer of material;
a second layer of material positioned external to the first layer of material and attached to the first layer of material to define a pocket space for receiving an electronics module; and
an elastomeric material provided between the first and second layers of material and arranged to tension the wearable article when worn, wherein the elastomeric material defines an open region in the pocket space for receiving the electronics module.

27. A wearable article as claimed in claim 26, wherein the elastomeric material comprises a first end and a second end, wherein the first and second ends are spaced apart from one another to define the open region in the pocket space.

28. A wearable article as claimed in claim 27, wherein the first end is attached to one or both of the first and second layers of material at a first edge of the pocket space, and the second end is attached to one or both of the second layers of material at a second edge of the pocket space.

29. A wearable article as claimed in claim 2, wherein the elastomeric material forms a continuous strip of material that extends from the first end to the second end.

30. A wearable article as claimed in claim 26, wherein the elastomeric material comprises an opening that forms the open region in the pocket space.

31. A wearable article as claimed in claim 30, wherein the elastomeric material forms a continuous loop of material.

32. A wearable article as claimed in claim 26, wherein the elastomeric material, when worn, extends around at least part of the wearer of the wearable article.

33. A wearable article as claimed in claim 26, wherein the first layer of material, second layer of material, and elastomeric material form a band which, when worn, extends around the circumference of at least part of the wearer of the wearable article.

34. A wearable article as claimed in claim 33, wherein the band is a waist band arranged to extend around the waist of the wearer.

35. A wearable article as claimed in claim 33, wherein the band is an arm band arranged to extend around the arm of the wearer.

36. A wearable article as claimed in claim 33, wherein the band is a chest band arranged to extend around the chest of the wearer.

37. A wearable article as claimed in claim 33, wherein the wearable article is a bra comprising a front portion, a back portion, and the band, wherein the band is an underband of the bra that extends from a lower margin of the front portion and the back portion of the bra.

38. A wearable article as claimed in claim 26, wherein the wearable article further comprises a sensing component, and wherein the electronics module is able to communicate with the sensing component through the open region when positioned in the pocket space.

39. A wearable article as claimed in claim 38, wherein the first layer of material comprises a recess arranged to receive at least part of the interface element of the electronics module so that the electronics module may communicate with the sensing component, wherein the recess is aligned with the open region formed by the elastomeric material.

40. A wearable article as claimed in claim 39, wherein the sensing component comprises a connection terminal for electrically connecting with the interface element of the electronics module, wherein the connection terminal is aligned with the recess in the first layer of the material.

41. A wearable article as claimed in claim 38, wherein the sensing component is attached to an inner surface of the first layer of material and the second layer of material is attached to an outer surface of the first layer of material.

42. A wearable article as claimed in claim 41, wherein sensing component has an outer surface facing the inner surface of the first layer of material and an inner surface facing towards the wearer when worn, the sensing component comprises a first conductive region provided on the inner surface and arranged to form an electrode for monitoring activity at a body surface of the wearer when worn, and a second conductive region provided on the outer surface and arranged to form a connection terminal for electrically connecting with the interface element of the electronics module when the interface element is positioned in the opening.

43. An assembly comprising an electronics module; and a wearable article as claimed in claim 26.

44. A method of manufacturing a wearable article, comprising:
providing a first layer of material and a second layer of material;
disposing an elastomeric material between the first and second layers of material, wherein the elastomeric material is arranged to tension the wearable article when worn; and
attaching the second layer of material to the first layer of material to form a pocket space for receiving an electronics module, wherein the elastomeric material defines an open region in the pocket space for receiving the electronics module.
